(19) United States
(12) Patent Application Publication
(10) Pub. No.: US 2008/0219935 A1
(43) Pub. Date: Sep. 11, 2008

(54) Phase Transitive Breath Care Products

(76) Inventors: Sang-Hoon Kwak, Daejeon (KR); Byung-Jun Kim, Daejeon (KR); Sang-Jin Kang, Seoul (KR); Sei-Young Yun, Seoul (KR); Hoo-Deok Kim, Daejeon (KR); Sang-Nyun Kim, Daejeon (KR)

Correspondence Address: BIRCH STEWART KOLASCH & BIRCH, PO BOX 747, FALLS CHURCH, VA, 22040-0747, US

(21) Appl. No.: 11/994,970

(22) PCT Filed: Jul. 7, 2002
(86) PCT No.: PCT/KR06/02650

§ 371 Date: Jan. 7, 2008

(30) Foreign Application Priority Data
Jul. 8, 2005 (KR) 10-2005-0061512

(51) Int. Cl. A61K 8/37; A61Q 11/00

(52) U.S. Cl. 424/49

(57) ABSTRACT

Disclosed herein is a breath freshener whose phase is changed depending on the condition of use. A breath freshener of W/O emulsion phase comprises a monoglyceride as a main base, a polymer, an effective component for breath freshening, and a solvent. A breath freshener of solution phase comprises a monoglyceride as a main base, a polyol, a polymer, an effective component for breath freshening, and a solvent. Since the breath fresheners are sufficiently flowable to spray when being sprayed into the oral cavity, they are easy to use. In addition, the breath fresheners are phase-changed into a cubic phase by moisture, e.g., saliva, after being sprayed into the oral cavity, resulting in an increase in the viscosity and adhesiveness of the breath fresheners. Therefore, the effective breath freshening ingredient is slowly released from the breath fresheners, and as a result, continuous breath freshening effects are achieved.

### TECHNICAL FIELD

The present invention relates to a breath freshener whose phase is changed depending on the condition of use, and more specifically to a formulation whose phase is changed into a cubic phase by moisture, e.g., saliva, present in oral cavity after being applied thereto so that the formulation can be adhered and fixed to oral cavity, and that allow a drug to be slowly released into oral cavity while being adhered to oral cavity due to the phase change, thereby exerting breath freshening effects.

### BACKGROUND ART

The three major functions of the oral cavity in humans are mastication, sound modulation for speech and aesthetic function. Healthy oral cavity can be defined as a state of oral tissues in which the main functions are in harmony with one another without any damage to social activities and mental functions as well as physical health. Oral health is mainly destroyed by oral diseases, such as dental caries and periodontal diseases. Various factors, such as coloration, may cause damage to the oral cavity in terms of beauty and may deteriorate the aesthetic function of the oral cavity. Further, other oral diseases and gastrointestinal diseases create a breath malodor, which often causes an obstacle in social activity.

Recent social developments have increased the scope of personal relationships and social activities and developed personal sanitary concepts. Under such circumstances, recognition for a breath malodor has gradually increased and there is thus a growing tendency to actively manage a breath malodor. In this connection, a great deal of research has been conducted on treatments effective for the removal of a breath malodor and blends of toothpastes, mouthwashes, capsules and patches with pharmacologically active agents in Korea and other countries.

Many techniques associated with breath fresheners have been developed. For example, toothpastes and mouthwashes comprising an antibacterial agent against breath malodor-causing bacteria and an ingredient for masking a breath malodor-causing substance have been investigated (U.S. Pat. Nos. 6,251,372 and 6,197,288). However, these formulations have limitations in their use for producing and maintaining breath freshening effects. For example, the toothpastes have a disadvantage in that the efficacy of the active ingredients cannot be maintained any longer after toothbrushing.

To overcome these disadvantages, attempts have been made to enhance the delivery effects of drugs by applying polymers to the drugs, as taught in U.S. Pat. Nos. 5,466,437 and 6,682,722. However, there still remains a disadvantage in that the use of ointment formulations, such as toothpastes, requires toothbrushing and water rinsing, which involve limitations in place.

Conventional breath freshening products include toothpastes, mouthwashes, chewing gums, mouth sprays, breath films, and the like. Toothpastes and mouthwashes have limitations in place of use. Mouth sprays and breath films have a short duration of their efficacy, causing a limited feeling of efficacy. Particularly, since breath films undergo shrinkage and aggregation in response to ambient temperature and humidity, they have an additional problem during distribution.

Disclosure

Technical Problem

Therefore, the present invention has been made in view of the above problems of the conventional breath fresheners, and it is an object of the present invention to provide novel types of spray-type breath fresheners that are convenient to carry and easy to use and whose phase is changed into a cubic phase by moisture, such as saliva, present in the oral cavity after being sprayed into the oral cavity, so that an effective breath freshening ingredient is slowly and continuously released into the oral cavity, thereby maintaining the breath freshening efficacy for a long time.

Technical Solution

In accordance with one aspect of the present invention for achieving the above object, there is provided a breath freshener of W/O emulsion phase comprising a monoglyceride as a main base, a polymer, an effective component for breath freshening, and a solvent.

In accordance with another aspect of the present invention, there is provided a breath freshener of solution phase comprising a monoglyceride as a main base, a polyol, a polymer, an effective component for breath freshening, and a solvent.

The present inventors have been earnestly and intensively conducted research to maintain the advantages and enhance the effects of conventional breath freshening products, and as a result, have developed formulations of breath fresheners having physical properties suitable for easily spraying into the oral cavity and having an extended duration of efficacy. Specifically, the present inventors have developed novel types of breath freshening systems whose phase is changed into a cubic phase by moisture, such as saliva, present in the oral cavity after being sprayed into the oral cavity, so that an effective breath freshening ingredient is slowly and continuously released into the oral cavity, thereby maintaining the breath freshening efficacy for a long time.

A W/O emulsion phase breath freshener composition according to a first embodiment of the present invention comprises a monoglyceride as a main base, a polymer, an effective breath freshening ingredient, and a solvent. After the W/O emulsion phase breath freshener composition is sprayed into the oral cavity, its phase is changed into a cubic phase so that the effective breath freshening ingredient is slowly released, thus achieving continuous removal or masking of a breath malodor.

A solution phase breath freshener composition according to a second embodiment of the present invention comprises a monoglyceride as a main base, a solvent, e.g., water or ethanol, for completely dissolving the monoglyceride, a surfactant, a polymer, and an effective breath freshening ingredient. Particularly, the gelling time of the monoglyceride can be controlled by varying the content of polyol having hydroxyl (—OH) group as the surfactant, and as a result, the active ingredient can be selectively delivered into the oral cavity and the throat over time. In addition, the phase stability of the breath freshener is improved by completely solubilizing the monoglyceride as a main base.

The present invention will now be described in greater detail.

The breath fresheners of the present invention use a monoglyceride, preferably glyceryl monooleate, as a main base. Since glyceryl monooleate undergoes phase change in response to temperature and moisture content, its structural shape and physical properties vary depending on the phase change, as already known in the art. According to the phase change of glyceryl monooleate, as glyceryl monooleate is phase changed into a lamellar, reverse micelle, cubic or reverse hexagonal phase, it loses its flowability and is changed from a flowable structure to a sticky solid structure. Based on these characteristics, as glyceryl monooleate is changed to a cubic phase in its structure with increasing temperature and moisture content when being sprayed into the oral cavity, it is solidified and loses its flowability.

The breath freshener formulations of the present invention have been developed by taking advantage of the fact that the phase of the glyceryl monooleate is changed depending on ambient conditions, such as temperature, moisture and pH. According to the formulations of the present invention, when the formulations are phase-changed after being sprayed into the oral cavity, the effective breath freshening ingredient is allowed to be slowly released from the tongue.

That is, the breath fresheners of the present invention are suitably flowable due to the use of the monoglyceride, preferably glyceryl monooleate, as a main base so that the breath fresheners are readily sprayed into the oral cavity. In addition, the breath fresheners of the present invention become rapidly viscous by the introduction of saliva thereinto so that a coating film is formed on the tongue and is well adhered to the tongue. As a result, the breath fresheners of the present invention are not readily diluted or lost under the internal conditions of the oral cavity. Optionally, the breath fresheners of the present invention may further comprise glyceryl trioleate to adjust an increase in the viscosity of the breath fresheners induced by saliva.

The monoglyceride used in the breath fresheners of the present invention may be glyceryl monooleate, glyceryl monolinoleate, glyceryl monoarachidonate, glyceryl monostearate, or a mixture thereof. The monoglyceride, preferably glyceryl monooleate, may be added in an amount of 3 to 95% by weight, based on the total weight of each of the compositions. For shape maintenance of the formulations, the glyceryl monooleate is preferably added in an amount of 25 to 70% by weight. When the glyceryl monooleate is added in an amount smaller than 3% by weight, effects of the phase change are negligible and it thus is difficult to effectively exert the intended functions. Meanwhile, when the glyceryl monooleate is added in an amount greater than 95% by weight, the phase change occurs during preparation and distribution, making it difficult to use.

In the W/O emulsion phase breath freshener according to the first embodiment of the present invention, an appropriate amount of a solvent is necessary to efficiently change the physical properties, particularly viscosity, of the breath freshener before and after spraying of the breath freshener into the oral cavity. That is, an increase in the viscosity of the breath freshener can be controlled by varying the content of the solvent, such as water or alcohol, in the main base. Particularly, when it is intended to apply some water-soluble, pharmacologically active ingredients to the breath freshener, the content of the solvent must be controlled in order to easily formulate the breath freshener. As the solvent suitable for the formulation, there can be used, for example, purified water, ethanol, glycerin, triglyceride, propylene glycol, triacetine or a mixture thereof. The solvent is present in an amount of about 1 to about 70% by weight and preferably 5 to 50% by weight, based on the total weight of the composition.

In the solution phase breath freshener according to the second embodiment of the present invention, an appropriate polyol is necessary to control the physical properties, and particularly adherence of the breath freshener, before and after spraying of the breath freshener into the oral cavity. The gelling time of the monoglyceride can be controlled by varying the content of the polyol having hydroxyl (—OH) group, and as a result, the active ingredient can be selectively delivered into the oral cavity and the throat over time. The polyol impedes the phase change of the glyceryl monooleate into a cubic phase to form a cubic of a sponge (network) structure. That is, an increased content of the polyol in the glyceryl monooleate as a main base results in decreased adhesion of the breath freshener, but causes a fast release of an effective breath freshening ingredient. Meanwhile, a decreased content of the polyol results in increased adhesion of the breath freshener, but causes a slow release of an effective breath freshening ingredient.

Examples of polyols suitable for use in the present invention include polyethylene glycol, propylene glycol, sorbitan monoester, and vegetable oils (e.g., castor oil and seed oils). The polyol is present in an amount of 0.01 to 20% by weight, preferably 0.1 to 15% by weight and more preferably 1 to 15% by weight, based on the total weight of the composition.

In the solution phase breath freshener according to the second embodiment of the present invention, water or ethanol may be used to solubilize the monoglyceride, preferably glyceryl monooleate. Since glyceryl monooleate is freely soluble in ethanol, ethanol is used as a basic solvent. A mixed solvent of water/ethanol can be used when it is intended to apply some water-soluble, pharmacologically active ingredients to the breath freshener. At this time, the water content is limited to 50% by weight or less with respect to the weight of the ethanol. If ethanol is used alone, it is used in an amount of about 1 to about 80% by weight and preferably 1 to 70% by weight, based on the total weight of the breath freshener. In the case where a mixed solvent of ethanol/water is used, since the addition of the water results in a decrease in the solubility of the glyceryl monooleate, the amount of the mixed solvent used is limited to the range of about 5 to about 90% by weight and preferably 10 to 80% by weight.

The breath fresheners of the present invention comprise a polymeric compound, i.e. a polymer, to control the physical properties of the main base (i.e. glyceryl monooleate). For example, the physical properties of the formulations can be varied by the addition of a polymer that is phase-changeable and has a high adhesive force, or a polymer that exhibits different behaviors depending on changes in temperature. The physical properties of the formulations may be varied despite the use of a small amount of the polymeric compound. In addition, the choice of the polymer is important because the content of moisture necessary for the change of flowability and the solidification varies depending on the amount of the polymeric compound added.

The polymer can be used in an amount of 0.01 to 30% by weight in the W/O emulsion phase breath freshener, and 0.01 to 20% by weight in the solution phase breath freshener in which the monoglyceride is solubilized. It is preferred to use the polymer in an amount of 0.1 to 10% by weight in order to make the breath fresheners sufficiently viscous to spray and to maintain the shape of the breath fresheners.

Examples of suitable polymers that can be used in the breath fresheners of the present invention include: non-ionic polymers, such as chitosan, polyvinyl alcohol, Poloxamer, polyvinyl pyrrolidone, polyvinyl pyrrolidone/vinyl acetate copolymers, hydroxypropyl cellulose, hydroxypropylmethyl cellulose, hydroxyethyl cellulose, hydroxypropylethyl cellulose, Polyox and salts thereof, anionic polymers, such as hyaluronic acid, carboxymethyl cellulose, carboxypropyl cellulose and salts thereof, xanthan gum, carrageenan gum, alginate gum, other polymers, such as karayan gum, arabic gum and salt derivatives thereof, gelatin, synthetic polymers, such as polyacrylic acid, Carbopol, polyquatemium-11, polyquatemium-39, polyalkylvinyl ether-maleic acid (PVM/MA) copolymers (Gantrez AN 119, AN 139, S-97) and mixtures thereof.

As the effective component for breath freshening used in the breath fresheners of the present invention, there can be used, for example, a disinfectant, a non-fermented sugar alcohol, a natural extract, an inorganic compound, an enzyme, or an enzyme inhibitor. Examples of disinfectants that can be used as the effective breath freshening ingredient in the breath fresheners of the present invention include triclosan, cetyl pyridium chloride, chlorhexidin gluconate, benzethonium chloride, and benzalkonium chloride. Examples of suitable non-fermented sugar alcohols include xylitol, sorbitol, erythritol, mannitol, maltitol, lactitol, palatinitol, palatinose, and oligosaccharide. Examples of suitable natural extracts include extracts of green tea, pancil, sanguinaria and Moutan Cortex. Examples of suitable inorganic compounds include zinc chloride, copper gluconate, tetrasodium pyrophosphate (TSPP), sodium acid pyrophosphate (SAPP), sodium hexametaphosphate (SHMP), sodium tripolyphosphate (STP), sodium potassium tripolyphosphate (SKTP), tetrapotassium pyrophosphate (TKPP), acidic sodium meta-polyphosphate, and acidic sodium polyphosphate. Examples of suitable enzymes include dextranase, glucose oxidase, glucose peroxidase, lactoperoxidase, glucanase, protease, and lysozyme. Examples of suitable enzyme inhibitors include peptide inhibitors and collagenase inhibitors, which are involved in the production of a breath malodor in the oral cavity.

For better breath freshening effects, these effective breath freshening ingredients may be used alone or as a mixture thereof. In the W/O emulsion phase breath freshener according to the first embodiment of the present invention, the effective breath freshening ingredient can be added in an amount of 0.001 to 50% by weight and preferably 0.01 to 30% by weight, based on the total weight of the composition. In the solution phase breath freshener according to the second embodiment of the present invention in which the monoglyceride is solubilized, the effective breath freshening ingredient can be added in an amount of 0.001 to 30% by weight and preferably 0.01 to 10% by weight, based on the total weight of the composition. If the effective breath freshening ingredient is a solid or powder, it can be previously dissolved or dispersed in water to prepare more homogeneous final breath fresheners.

The breath fresheners of the present invention are advantageous in stabilizing the effective breath freshening ingredient and are preferred in preventing deterioration of efficacy during distribution and storage, compared to conventional breath fresheners, such as toothpastes and mouthwashes. In addition, the breath fresheners of the present invention may further comprise a pH-adjusting agent having a function to adjust the pH of the breath freshening active ingredient as a stabilizer because the efficacy of the breath freshening active ingredient may vary as a function of pH. The stabilizer having a pH-adjusting function serves to inhibit variation in pH during storage and to maintain the pH value so as not to irritate the oral cavity. Examples of such stabilizers include organic acids, such as citric acid, malic acid, succinic acid, tartaric acid, formic acid and lactic acid, salts thereof, and mixtures thereof. The amount of the stabilizers used may vary depending on the kind and content of the effective breath freshening ingredient used.

In addition to the aforementioned ingredients, the breath fresheners of the present invention may further comprise at least one additive selected from flavors and sweetening agents to enhance feeling of use and impart a favorable taste to the breath fresheners. Representative flavors are peppermint, spearmint, menthol, citrus, herbs, and the like. Representative sweetening agents are stevia, amino acids, aspartame, sodium saccharin, and the like.

The novel types of breath freshening delivery systems comprising the above-mentioned ingredients offer improved feeling of use and increased efficacy.

Advantages of the breath fresheners according to the present invention are summarized below:

Firstly, when the breath fresheners of the present invention are sprayed into the oral cavity to deliver an effective breath freshening ingredient to the oral cavity, it is adhered and fixed to the tongue and teeth. Specifically, the introduction of moisture, e.g., saliva, into the oral cavity causes an increase in the viscosity of the breath fresheners, resulting in adherence and fixation of the breath fresheners to the oral cavity. The breath fresheners of the present invention are suitably flowable during spraying and are convenient to use. Since the breath fresheners of the present invention are phase-changed into a cubic phase by the action of saliva after being sprayed into the oral cavity, they are not readily diluted or lost by saliva.

Secondly, since the effective breath freshening ingredient is slowly released over a long period of time, the duration of efficacy can be extended. Specifically, after the breath fresheners become viscous due to the introduction of moisture, e.g., saliva, and are attached to the tongue, the effective breath freshening ingredient present in the breath fresheners is slowly released. This slow release of the effective breath freshening ingredient can increase leads to an increased contact time between the effective breath freshening ingredient and breath malodor sources.

Thirdly, in the solution phase breath freshener in which the monoglyceride is completely solubilized, the phase stability is improved, thus resulting in good low-temperature stability. In addition, the use of the polyol in the solution phase breath freshener enables control of the time required for the phase change, and as a result, the breath freshening active ingredient can be selectively delivered into the throat and the oral cavity over time.

### Best Mode

Hereinafter, the present invention will be explained in more detail with reference to the following preferred examples. The following examples are provided to assist in understanding of the present invention, and are not intended to limit the present invention.

### EXAMPLES 1 to 5 AND COMPARATIVE EXAMPLE 1

In accordance with each of the compositions indicated in Table 1, glyceryl monooleate and glyceryl monolinoleate were liquefied in a bath while heating to about 50° C., and then the liquid was mixed with the oil-soluble ingredients to obtain a homogeneous solution. The water-soluble ingredients were mixed with purified water and ethanol to obtain a homogeneous solution. The two solutions thus obtained were mixed together to prepare a W/O emulsion phase breath freshener. The values shown in Table 1 indicate % by weight (w/w in 100 g).

TABLE 1

| | Comparative | | | | | |
|---|---|---|---|---|---|---|
| Ingredient | Example 1 | Example 1 | Example 2 | Example 3 | Example 4 | Example 5 |
| Glyceryl monooleate | — | 45.0 | 35.0 | 40.0 | 20 | 10 |
| Glyceryl monolinoleate | — | — | 15.0 | — | 30 | 35 |
| Glycerin | 20.0 | 10.0 | 10.0 | 5.0 | 8.0 | 7.0 |
| Xylitol | 5.0 | 5.0 | — | 8.0 | 5.0 | 3.0 |
| Dextranase | — | — | 0.1 | — | — | 0.05 |
| Green tea extract | 0.2 | 0.2 | — | — | — | 0.1 |
| Cetyl pyridium chloride | 0.05 | 0.05 | — | — | 0.05 | — |
| Polyvinyl pyrrolidone | — | — | 2.0 | — | — | 0.5 |
| Poloxamer | 5.0 | 0.1 | 0.1 | 3.0 | 0.1 | 0.5 |
| Citric acid | 0.1 | — | 0.1 | — | 0.05 | 0.05 |
| Sodium citrate | 0.5 | — | 0.5 | — | 0.25 | 0.25 |
| Ethanol | 30.0 | 30.0 | 20.0 | 35.0 | 20.0 | 30.0 |
| Flavor | 5.0 | 5.0 | 5.0 | 5.0 | 5.0 | 5.0 |
| Purified water | To 100 | To 100 | To 100 | To 100 | To 100 | To 100 |

### EXAMPLES 6 AND 7 AND COMPARATIVE EXAMPLE 2

In accordance with each of the compositions indicated in Table 2, glyceryl monooleate was liquefied in a bath while heating to about 50° C., and then the liquid was mixed with the oil-soluble ingredients to obtain a homogeneous solution. The water-soluble ingredients were mixed with purified water and ethanol to obtain a homogeneous solution. The two solutions thus obtained were mixed together to prepare a W/O emulsion phase breath freshener.

### EXAMPLES 8 TO 11

In accordance with each of the compositions indicated in Table 2, glyceryl monooleate was completely dissolved in ethanol in a bath at room temperature, and then the polyol and the oil-soluble ingredients were dissolved in the solution. Purified water and the water-soluble ingredients were mixed together to prepare a solubilized breath freshener. The values shown in Table 2 indicate % by weight (w/w in 100 g).

TABLE 2

| | Comparative | | | | | Example | Example |
|---|---|---|---|---|---|---|---|
| Ingredient | Example 2 | Example 6 | Example 7 | Example 8 | Example 9 | 10 | 11 |
| Glyceryl | — | 45.0 | 50.0 | 45.0 | 50.0 | 50.0 | 50.0 |
| monooleate | | | | | | | |
| Propylene glycol | 15.0 | 10.0 | 10.0 | 10.0 | 10.0 | 5.0 | 2.0 |
| Xylitol | 5.0 | 5.0 | — | 5.0 | — | — | — |
| Dextranase | — | — | 0.1 | — | 0.1 | 0.1 | 0.1 |
| Green tea extract | 0.2 | 0.2 | — | 0.2 | — | — | — |
| Cetyl pyridium | 0.05 | 0.05 | — | 0.05 | — | — | — |
| chloride | | | | | | | |
| Polyvinyl | — | — | 2.0 | — | 2.0 | 2.0 | 2.0 |
| pyrrolidone | | | | | | | |
| Poloxamer | 5.0 | 0.1 | 0.1 | 0.1 | 0.1 | 0.1 | 0.1 |
| Citric acid | 0.1 | — | 0.1 | — | 0.1 | 0.1 | 0.1 |
| Sodium citrate | 0.5 | — | 0.5 | — | 0.5 | 0.5 | 0.5 |
| Ethanol | 35.0 | 30.0 | 20.0 | 30.0 | 20.0 | 25.0 | 28.0 |
| Flavor | 5.0 | 5.0 | 5.0 | 5.0 | 5.0 | 5.0 | 5.0 |
| Purified water | To 100 | To 100 | To 100 | To 100 | To 100 | To 100 | To 100 |

Tests for Effects

(1) Measurement of Change in Viscosity

Changes in the viscosity of the formulations prepared in Examples 1 to 11 and Comparative Examples 1 and 2 before and after the introduction of saliva were measured using a Brookfield RVT viscometer. The results are shown in Tables 3 and 4. As the saliva, artificial saliva was used. The saliva was introduced in an amount of 100% by weight, relative to the weight of each of the formulations.

TABLE 3

| | Comparative | | | | | |
|---|---|---|---|---|---|---|
| Viscosity (cps) | Example 1 | Example 1 | Example 2 | Example 3 | Example 4 | Example 5 |
| Viscosity before | 5000 ± 500 | 5000 ± 400 | 5800 ± 600 | 4500 ± 500 | 5500 ± 500 | 5200 ± 500 |
| introduction of saliva | | | | | | |
| Viscosity after | 4500 ± 400 | 19500 ± 500 | 23800 ± 500 | 22000 ± 800 | 25000 ± 1000 | 25200 ± 600 |
| introduction of saliva | | | | | | |

TABLE 4

| | Comparative | | | | | Example | Example |
|---|---|---|---|---|---|---|---|
| Viscosity (cps) | Example 2 | Example 6 | Example 7 | Example 8 | Example 9 | 10 | 11 |
| Viscosity before | 2500 ± 500 | 5000 ± 400 | 5800 ± 600 | 5000 ± 400 | 5500 ± 500 | 5100 ± 500 | 5200 ± 500 |
| introduction of saliva | | | | | | | |
| Viscosity after | 2500 ± 400 | 19500 ± 500 | 23800 ± 500 | 19500 ± 500 | 25000 ± 1000 | 25500 ± 600 | 25200 ± 600 |
| introduction of saliva | | | | | | | |

As can be seen from the data shown in Tables 3 and 4, the breath fresheners containing glyceryl monooleate according to the present invention showed an increase in viscosity and were hardened due to the introduction of moisture, irrespective of their formulations.

(2) Evaluation of Dissolution Rate and Remained Amount of Breath Fresheners

The maintenance time of the phase-changed formulations after spraying into the oral cavity was evaluated by flowing artificial saliva at a rate of 1 ml/min., and measuring the time required for complete dissolution of the formulations. The results are shown in Table 5.

TABLE 5

| | Comparative | | | | | |
|---|---|---|---|---|---|---|
| Time (sec.) | Example 1 | Example 1 | Example 2 | Example 3 | Example 4 | Example 5 |
| Time required for | <10 | 1540 | 1620 | 1580 | 1730 | 1750 |
| complete dissolution | | | | | | |

The results of Table 5 reveal that the breath fresheners prepared in Examples 1 to 5 required a considerably short time for complete dissolution in comparison with the formulation prepared in Comparative Example 1.

(3) Evaluation of Phase Stability

The breath fresheners prepared in Examples 6 to 11 and Comparative Example 2 were placed in thermostatic baths at room temperature, 40° C., 50° C., 60° C., 0° C. and −10° C. The phase stability of the breath fresheners was evaluated during storage for 3 months. The results are shown in Table 6. The symbol ‘⊚’ indicates good phase stability, and the symbol ‘x’ indicates that phase separation occurred.

TABLE 6

| | Comparative | | | | | Example | Example |
|---|---|---|---|---|---|---|---|
| Viscosity (cps) | Example 2 | Example 6 | Example 7 | Example 8 | Example 9 | 10 | 11 |
| −10° C. | X | X | X | ⊚ | ⊚ | ⊚ | ⊚ |
| 0° C. | X | X | X | ⊚ | ⊚ | ⊚ | ⊚ |
| Room Temp. | ⊚ | ⊚ | ⊚ | ⊚ | ⊚ | ⊚ | ⊚ |
| 40° C. | ⊚ | ⊚ | ⊚ | ⊚ | ⊚ | ⊚ | ⊚ |
| 50° C. | ⊚ | ⊚ | ⊚ | ⊚ | ⊚ | ⊚ | ⊚ |
| 60° C. | ⊚ | ⊚ | ⊚ | ⊚ | ⊚ | ⊚ | ⊚ |

As is evident from the results of Table 6, the W/O emulsion phase breath fresheners showed poor low-temperature stability, whereas the breath fresheners in which glyceryl monooleate was solubilized showed good low-temperature stability.

(4) Evaluation of Time Required for Phase Change

0.2 g of each of the breath fresheners prepared in Examples 9 to 11 was dropped on a slide glass, covered with a cover glass, and placed on a polarizing microscope. Next, 1 g of artificial saliva was dropped on one end of the cover glass using a spoid. Changes in the crystallinity of the breath fresheners were observed using a digital camera at continuous shooting of eight frames per second to determine the time required for the phase change. The results are shown in Table 7.

TABLE 7

| | Example No. | | |
|---|---|---|---|
| | Example 9 | Example 10 | Example 11 |
| Time required for phase | 7.8 ± 0.45 | 4.7 ± 0.22 | 2.9 ± 0.13 |
| change (sec.) | | | |

From the results of Table 7, it could be confirmed that the time required for the phase change was extended with increasing content of the polyol.

(5) Evaluation for Breath Freshening Effects

The initial breath malodor intensity of a total of sixty healthy men and women aged 20-40 was evaluated. The subjects were divided into six groups (10 per group) who had similar initial breath malodor intensities. The breath malodor intensity was evaluated using a 10-point scale by an expert panel test (four panels). According to the 10-point scale, 1 point indicates that no breath malodor was produced, 5 point indicates that a breath malodor was produced but was unpleasant, and 9 point indicates that an unendurable and unpleasant breath malodor was produced. The breath malodor intensities were evaluated 2 and 30 minutes after spraying of the formulations twice. During the test, no food and water were provided but conversation was allowed to the subjects. The results are shown in Table 8.

TABLE 8

| | Comparative | | | | | |
|---|---|---|---|---|---|---|
| Point | Example 1 | Example 1 | Example 2 | Example 3 | Example 4 | Example 5 |
| 2 minutes | 4.0 ± 0.45 | 4.1 ± 0.25 | 4.2 ± 0.35 | 3.5 ± 0.25 | 3.4 ± 0.45 | 3.9 ± 0.45 |
| after use | | | | | | |
| 30 minutes | 6.7 ± 0.35 | 5.2 ± 0.30 | 5.3 ± 0.40 | 4.5 ± 0.25 | 4.8 ± 0.35 | 4.0 ± 0.45 |
| after use | | | | | | |

As is evident from the results of Table 8, there was a distinct difference in the efficacy between the breath fresheners prepared in Examples 1 to 5 and the breath freshener prepared in Comparative Example 1. Two minutes after spraying, the breath freshener prepared in Comparative Example 1 showed better results than the breath fresheners prepared in Examples 1 and 2. However, thirty minutes after spraying, the breath fresheners prepared in Examples 1 and 2 showed much better results than the breath freshener prepared in Comparative Example 1. These results are because the breath fresheners prepared in Examples 1 to 5 slowly released the effective breath freshening ingredients through the phase change after spraying of the breath fresheners into the oral cavity, compared to the breath freshener prepared in Comparative Example 1.

By a significant difference test, it was confirmed that all the breath fresheners prepared in Examples 1 to 5 and Comparative Example 1 were within a significant level (p<0.05).

(6) Evaluation of Convenience of Use

The breath freshener samples prepared in Example 1 and Comparative Example 1 were given to a total of thirty healthy adults aged 20-40. After the samples were used in the same manner as conventional mouth sprays, the taste and convenience of use of the samples were scored by a 5-point scale. The results are shown in Table 9.

TABLE 9

| | | Comparative Example 1 |
|---|---|---|
| | Example 1 (n = 30) | (n = 30) |
| Convenience of use | 4.1 | 3.6 |

From the results of Table 9, it could be confirmed that the breath freshener prepared in Example 1 offered a good feeling of use, compared to the breath freshener prepared in Comparative Example 1 (p<0.05).

### INDUSTRIAL APPLICABILITY

As apparent from the above description, according to the breath fresheners of the present invention, a monoglyceride, preferably glyceryl monooleate, is used as a main base and a polymeric compound is used for the purpose of controlling the shape or adhesion to the oral cavity. Since the breath fresheners of the present invention are sufficiently flowable to spray when being sprayed into the oral cavity, they are easy to use. In addition, the breath fresheners of the present invention are phase-changed into a cubic phase by moisture, e.g., saliva, after being sprayed into the oral cavity, resulting in an increase in the viscosity and adhesiveness of the breath fresheners. Therefore, an effective breath freshening ingredient is slowly released from the breath fresheners, and as a result, continuous breath freshening effects are achieved.

Although the preferred embodiments of the present invention have been disclosed for illustrative purposes, those skilled in the art will appreciate that various modifications, additions and substitutions are possible, without departing from the scope and spirit of the invention as disclosed in the accompanying claims.

1. A breath freshener of W/O emulsion phase comprising a monoglyceride as a main base, a polymer, an effective component for breath freshening, and a solvent.

2. A breath freshener of solution phase comprising a monoglyceride as a main base, a polyol, a polymer, an effective component for breath freshening, and a solvent.

3. The breath freshener according to claim 1 or 2, wherein the monoglyceride is glyceryl monooleate, glyceryl monolinoleate, glyceryl monoarachidonate, glyceryl monostearate, or a mixture thereof.

4. The breath freshener according to claim 1 or 2, wherein the monoglyceride is present in an amount of 3 to 95% by weight, based on the total weight of the composition.

5. The breath freshener according to claim 2, wherein the polyol is polyethylene glycol, propylene glycol, sorbitan monoester, a vegetable oil, or a mixture thereof.

6. The breath freshener according to claim 2, wherein a gelling time of the monoglyceride is controlled by the content of the polyol.

7. The breath freshener according to claim 2, wherein the polyol is present in an amount of 0.01 to 20% by weight, based on the total weight of the composition.

8. The breath freshener according to claim 7, wherein the polyol is present in an amount of 1 to 15% by weight, based on the total weight of the composition.

9. The breath freshener according to claim 1 or 2, wherein the polymer is chitosan, polyvinyl alcohol, Poloxamer, polyvinyl pyrrolidone, a polyvinyl pyrrolidone/vinyl acetate copolymer, hydroxypropyl cellulose, hydroxypropylmethyl cellulose, hydroxyethyl cellulose, hydroxypropylethyl cellulose, Polyox and a salt thereof, hyaluronic acid, carboxymethyl cellulose, carboxypropyl cellulose, xanthan gum, carrageenan gum, alginate gum, karayan gum, arabic gum and a salt derivative thereof, gelatin, polyacrylic acid, Carbopol, polyquatemium-11, polyquatemium-39, a polyalkylvinyl ether-maleic acid (PVM/MA) copolymer, or a mixture thereof.

10. The breath freshener according to claim 1, wherein the polymer is present in an amount of 0.01 to 30% by weight, based on the total weight of the composition.

11. The breath freshener according to claim 2, wherein the polymer is present in an amount of 0.01 to 20% by weight, based on the total weight of the composition.

12. The breath freshener according to claim 1, wherein the solvent is purified water, ethanol, glycerin, triglyceride, propylene glycol, triacetine, or a mixture thereof, and is present in an amount of 1 to 70% by weight, based on the total weight of the composition.

13. The breath freshener according to claim 2, wherein the solvent is ethanol or a mixed solvent of water/ethanol, and is present in an amount of 5 to 90% by weight, based on the total weight of the composition.

14. The breath freshener according to claim 1 or 2, wherein the effective component for breath freshening is a disinfectant, a non-fermented sugar alcohol, a natural extract, an inorganic compound, an enzyme, an enzyme inhibitor, or a mixture thereof.

15. The breath freshener according to claim 14, wherein the disinfectant is triclosan, cetyl pyridium chloride, chlorhexidin gluconate, benzethonium chloride, benzalkonium chloride, or a mixture thereof.

16. The breath freshener according to claim 14, wherein the non-fermented sugar alcohol is xylitol, sorbitol, erythritol, mannitol, maltitol, lactitol, palatinitol, palatinose, oligosaccharide, or a mixture thereof.

17. The breath freshener according to claim 14, wherein the natural extract is green tea extract, pancil extract, sanguinaria extract, Moutan Cortex extract, or a mixture thereof.

18. The breath freshener according to claim 14, wherein the inorganic compound is zinc chloride, copper gluconate, tetrasodium pyrophosphate (TSPP), sodium acid pyrophosphate (SAPP), sodium hexametaphosphate (SHMP), sodium tripolyphosphate (STP), sodium potassium tripolyphosphate (SKTP), tetrapotassium pyrophosphate (TKPP), acidic sodium meta-polyphosphate, acidic sodium polyphosphate, or a mixture thereof.

19. The breath freshener according to claim 14, wherein the enzyme is dextranase, glucose oxidase, glucose peroxidase, lactoperoxidase, glucanase, protease, lysozyme, or a mixture thereof.

20. The breath freshener according to claim 14, wherein the enzyme inhibitor is a peptide inhibitor, a collagenase inhibitor, or a mixture thereof.

21. The breath freshener according to claim 1, wherein the effective component for breath freshening is present in an amount of 0.001 to 50% by weight, based on the total weight of the composition.

22. The breath freshener according to claim 2, wherein the effective component for breath freshening is present in an amount of 0.001 to 30% by weight, based on the total weight of the composition.
